(19) United States
(12) Patent Application Publication
(10) Pub. No.: US 2026/0306035 A1
(43) Pub. Date: Oct. 8, 2026

(54) AN ELECTROMAGNETIC RADIATION POLY-CAPILLARY BLOCK FILTER AND APPLICATIONS THEREOF

(71) Applicant: GOYA DENTAL PTY LTD, West Gosford (AU)

(72) Inventor: Kyle Andrew Mervin, East Gosford (AU)

(73) Assignee: GOYA DENTAL PTY LTD, West Gosford (AU)

(21) Appl. No.: 19/252,448

(22) Filed: Jun. 27, 2025

Related U.S. Application Data
(63) Continuation of application No. 18/247,578, filed on Sep. 15, 2021, now Pat. No. 12,364,446.

(30) Foreign Application Priority Data
Oct. 13, 2020 (AU) 2020903716

(51) Int. Cl. A61B 6/06; A61B 6/40

(52) U.S. Cl. CPC A61B 6/06; A61B 6/4007

(57) ABSTRACT

An electromagnetic radiation collimator comprising: a radiopaque elongated housing having a first end and a second end, wherein the housing houses an array of parallel radiolucent beam paths, wherein each of the beam paths extend from the first end to the second end, wherein each of the beam paths has an entrance aperture at the first end, and an exit aperture at the second end; the entrance aperture through which source electromagnetic radiation passes into the beam paths, wherein the beam paths emits an electromagnetic radiation beam from the exit aperture when the source electromagnetic radiation beams are in line with the longitudinal axis of the beam path or having a predetermined angle of deviation from the longitudinal axis of the beam path.

### TECHNICAL FIELD

Diagnostic imaging with x-ray radiation based on the notion, that an x-ray originating from a source travels along a straight-line path between the source and the tissue being surveyed, and then on to the sensor. The relative radio opacity of the specimen along that path can be determined by the strength of the beam that reaches the sensor, compared with the strength of the beam that leaves the source. Adjacent and parallel paths can then be used to form a 2 dimensional image.

Conventional designs are hampered by the scatter produced when stray x-rays interact with various materials within their straight-line pathway between the x-ray source and the sensor. Additionally, the x-ray source (focal spot) more approximates a point source producing x-ray photons that propagate in divergent paths and cannot by definition produce beams that are parallel.

The present invention is a device that utilizes the properties of x-ray radiation to produce a series of beams that are approaching parallel, such that the radio-lucency/radio-opacity of specific parallel pathways through the tissue can be accurately determined and simply measured.

The present invention is a filter that can be used to condition the beam emerging from an x-ray (or other EM radiation emitting) tube into a series of distinct beams closely approximating parallel. The device is constructed out of radio-opaque material, which creates a physical barrier to the passage of unwanted/non-parallel beams, whilst selectively allowing beams that are parallel (or nearing parallel relative to each other) to emerge from the diagnostic end of the device. Thus allowing for diagnostic imaging to be based on a parallel beams. The present device is designed to either replace the conventional collimator or retrofit an x-ray unit between the collimator and specimen to further modify the diagnostic beam.

### BACKGROUND

A conventional x-ray tube has three elements responsible for the composition of a diagnostic beam. The focal spot, which generates the primary x-rays, a filter which selectively removes useless x-rays, and becomes a secondary source of x-rays, and a collimator which provides a physical, radio-opaque barrier to divergent beams.

Typically, collimation of X-rays enhances the image quality of an X-ray imaging system. Collimation is important for image quality as collimation of an X-ray source reduces the amount of scattered X-ray photons which reach the X-ray sensor elements, after having passed through the subject matter being imaged. These scattered X-ray photons would otherwise contribute to the reading from the sensor elements and reduce the overall contrast in the X-ray image because they do not convey the same relevant diagnostic information as the unscattered or parallel X-ray photons that have passed directly from the X-ray source to the sensor element. Scattered photons are responsible for the haziness often associated with radiographs.

Conventional X-ray collimators uses an anti-scatter grid, which can be a two-dimensional grid, positioned directly in front of the sensor and the grid serves to absorb or block photons emanating with a large angle. These grids are often grid structures composed of high-density metals. A possible limitation of such grid structures is that the photons emanating with lower angles may still be considered a large angle of deviation from the longitudinal axis of the path of the beam, thereby still considered as allowing scattered photons.

An example of such a two-dimensional X-ray source is provided in WO2011/017645 apparatus for producing X-rays for use in imaging. Known collimation and lensing methods are not so useful for collimating a two-dimensional array of X-ray sources and it is an aim of the embodiments of the present invention to at least partially mitigate the disadvantages of known X-ray collimation methods and to provide a means of collimating X-rays whereby multiple collimating elements or holes allow for the passage of x-ray photons derived from a scatter medium distinct from the primary x-ray source (focal spot).

For example, Australian Patent No. 2015225939 describes an X-ray collimator and a method of obtaining an X-ray image and finds utility in the collimation of X-ray Bremsstrahlung radiation, where the X-ray source comprises a plurality of X-ray sources arranged in a two-dimensional array. Another example, U.S. Pat. No. 8,774,351 describes an apparatus and method for X-ray imaging a human patient. However, these examples may use collimators that have relatively large beam path sizes that may not significantly filter out non-diagnostically valuable photons for medical imaging, thereby may produce a relatively hazy or a relatively low-resolution image of the subject region of interest. While examples of collimating X-ray radiation have been discussed in detail, this principle is also applicable to collimation of other medically useful electromagnetic radiation. That is, even if it is another type of electromagnetic radiation, by not significantly filtering out non-diagnostically valuable photons, it may also produce the similar result of producing low-resolution image of the subject region of interest.

In light of the aforementioned disadvantages, there exists a long-felt need to provide an improved electromagnetic radiation collimator for ultimately producing high resolution medical images when diagnostically valuable photons are selectively passed through when the beams are either in line with the longitudinal axis of the beam path or a relatively minute angle of deviation from the longitudinal axis of the beam path.

Any discussion of the prior art throughout the specification should in no way be considered as an admission that such prior art is widely known or forms part of common general knowledge in the field.

### SUMMARY

### Problems to be Solved

It may be advantageous to provide an electromagnetic collimation device, that facilitates accurate 2-dimensional measurements of target tissues.

It may be advantageous to provide an electromagnetic radiation collimator that can be retrofitted to existing X-ray machines or other electromagnetic radiation generating machines.

It may be an advantage to provide an array of beam paths in the elongate housing, in which each of the beam paths has a width suitable for use for a particular type of electromagnetic radiation.

It may be an advantage to provide an elongate radio-opaque housing that can house an array of radiolucent tubes.

It may be an advantage to provide a central tube for engaging with a spinning motor to allow the elongate housing to spin.

It may be an advantage to provide an array of beam paths, each having a sufficient length so that the propagated electromagnetic radiation through the beam path will emit a beam in a narrow angle cone.

It may be an advantage to provide a second collimator similar to the first collimator between the subject region of interest and the detector.

It is an object of the present invention to overcome or ameliorate at least one of the disadvantages of the prior art, or to provide a useful alternative.

### Means for Solving the Problem

A first aspect of the present invention may relate to an electromagnetic radiation collimator comprising: a radiopaque elongated housing having a first end and a second end, wherein the housing houses an array of parallel radiolucent beam paths, wherein each of the beam paths extend from the first end to the second end, wherein each of the beam paths has an entrance aperture at the first end, and an exit aperture at the second end. The entrance aperture through which source electromagnetic radiation passes into the beam paths, wherein the beam paths emits an electromagnetic radiation beam from the exit aperture when the source electromagnetic radiation beams are in line with the longitudinal axis of the beam path or having a predetermined angle of deviation from the longitudinal axis of the beam path.

Preferably, the array of parallel radiolucent beam paths is an array of parallel radiolucent tubes, wherein each of the tubes defines a lumen for providing the beam path, and wherein the radiolucent tubes are embedded in the elongate housing.

Preferably, the elongate housing is composed of a linear series of alternating first and second layers offset relative to each other, wherein the first layer comprises a first set of apertures each having a first shape, and the second layer comprises a second set of apertures each having a second shape, wherein the first shape and the second shape are partially intersecting such that the series of intersected shapes defines a lumen for providing the beam path. Preferably, the elongate housing is composed of a single block with patent lumen running parallel to the long axis of the block, with the spatial separation of the individual lumen defining the resolution of the diagnostic image.

Alternatively, the elongate housing may be composed of segments arranged in series, with the lumen embedded in each segment offset, so that the beam that emerges from the device is further refined.

Preferably, the first elongated housing has a length longer than the second elongated housing, the width of the entrance aperture of the first elongated housing is smaller than the width of the entrance aperture of the second elongated housing.

Preferably, the predetermined angle of deviation from the longitudinal axis of the beam path is less than 10°. More preferably, the predetermined angle of deviation from the longitudinal axis of the tubes is less than 1°. Desirably, the predetermined angle of deviation from the longitudinal axis of the tubes is 0°.

Preferably, the material of the elongate housing is one selected from the group of: aluminium, zirconia, titanium or lead or any particulate matter that is radio-opaque and can be bound in a resin for 3-dimensional printing or production purposes.

Preferably, the elongate housing comprises at least a groove, wherein the grooves is adapted to engage to a spinning motor for spinning or oscillating the elongate housing.

Preferably, each of the grooves is 3 mm deep, and a width of 5 mm wide.

Preferably, the width of each beam paths is 100 microns.

Preferably, the electromagnetic radiation is one selected from the group of: X-ray radiation, and UV-C radiation.

Preferably, the width of the entrance and exit apertures for use with X-ray radiation are relatively smaller than width of the entrance and exit apertures for use with microwave radiation.

In a second aspect of the present invention may relate to an electromagnetic radiation imaging device adapted for use with two electromagnetic radiation collimators according to the first aspect, wherein the device comprises: an electromagnetic radiation source for generating electromagnetic radiation. The electromagnetic radiation collimator positioned between the radiation source and the subject region, such that the subject region is subjected to parallel electromagnetic radiation from the collimator. The second electromagnetic radiation collimator positioned between the subject region and an electromagnetic radiation detector, wherein the second electromagnetic radiation collimator is adapted for filtering out scattered radiation after propagating through the subject region such that the radiation that reaches the diagnostic sensor is a more accurate indication of the radio-lucent properties of the subject within the path of the diagnostic beam.

Preferably, for X-ray radiation, the primary electromagnetic radiation source is a rotating anode X-ray source offset from the longitudinal axis of the device, such that there are no direct x-ray photons between the source and the sensor.

Preferably, for UV-C radiation, the primary electromagnetic radiation source is a UV lamp.

Preferably, the electromagnetic radiation detector is an X-ray film or x-ray sensor for X-ray radiation, or a UV-C light sensor.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 illustrates the device mounted to and in use with an electromagnetic radiation generation apparatus such that electromagnetic radiation that enters the device in line with the beam paths of the device are emitted from the exit end of the device in parallel lines.

FIG. 2 illustrates a schematic of a first device positioned between the electromagnetic radiation generation apparatus and the subject, and a second similar device positioned between the subject and the detector. The first and second device allows electromagnetic radiation that are in line with the beam paths of the devices to traverse through and filters away the scattered beams such that only parallel radiation reaches the detector.

FIG. 3 illustrates a vertical cross-sectional view of the device showing the array of entrance apertures at the first end. It may be appreciated that it may also be a vertical cross-sectional view of the device showing the array of exit apertures at the second end. FIG. 4 illustrates a perspective view of the device showing the grooves that are adapted to connect to a motor which allows the device to be rotated, when in use.

FIG. 5 illustrates a beam path of width C¹-D¹ with length A-E. Between the beam path and an adjacent beam path, the material of the device may have a thickness of C¹-C² or D¹-D².

FIG. 6 illustrates a beam path of width C-D with variable lengths A-E³, or A-E¹, or A-E² showing the allowable angle of deviation from the longitudinal axis of the beam paths from the electromagnetic radiation source.

FIG. 7 illustrates a side view of the elongate housing as an integral piece (top) or composed of block or segments (bottom) joined together to similarly form an integral piece.

FIG. 8 illustrates the device mounted to and in use with another embodiment of an electromagnetic radiation generation apparatus such that electromagnetic radiation that enters the device in line with the beam paths of the device are emitted from the exit end of the device in parallel lines.

FIG. 9 illustrates a conventional schematic of an X-ray tube showing the parts and the X-ray photon beam emitted from the window of the X-ray tube.

FIG. 10 illustrates a schematic of a parallel imaging field with the x, y, and z axis.

FIG. 11A illustrates the array of apertures in black circles in which the device or filter is static.

FIG. 11B illustrates the projection of the array of apertures of FIG. 11A when static.

FIG. 12A illustrates the same array of apertures in black circles of FIG. 11A.

FIG. 12B illustrates the projection of the array of apertures of FIG. 12A when rotating.

FIG. 13 illustrates a superimposition of lumens from blocks or segments that are equidistant from the central axis producing a projection of concentric circles.

FIG. 14 illustrates the projection of the rotation of the device with the superimposition of lumens are staggered radially from the central axis.

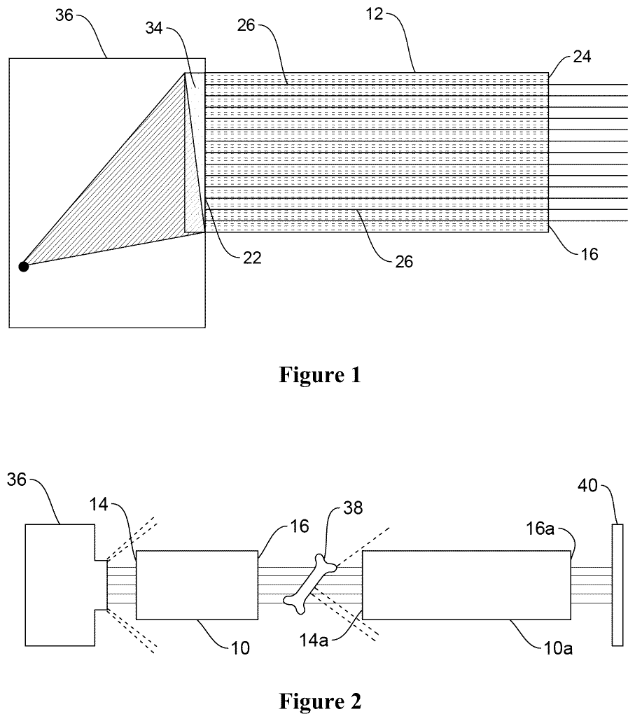

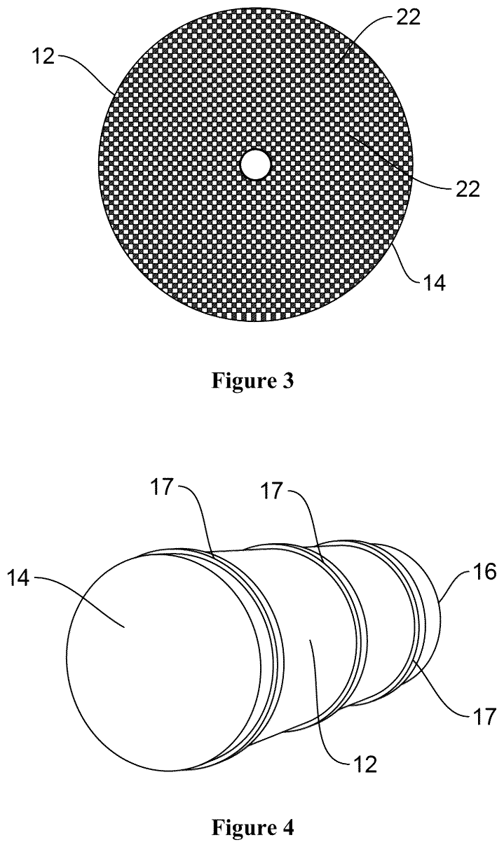

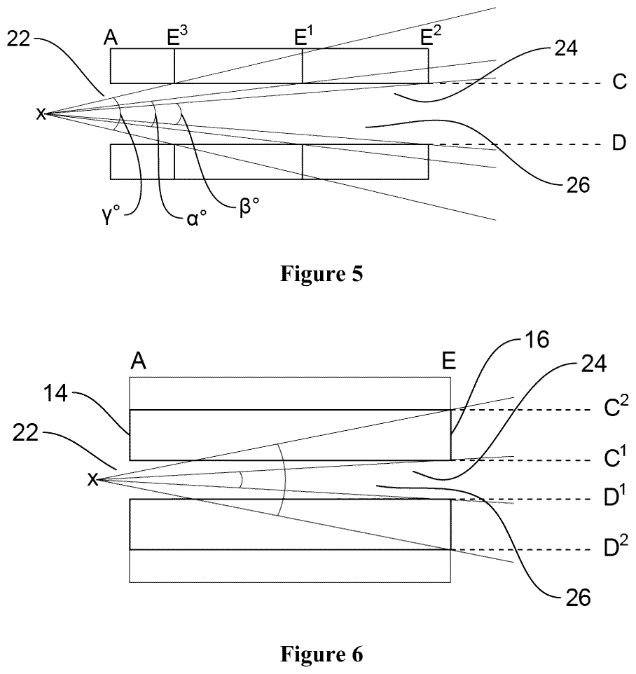

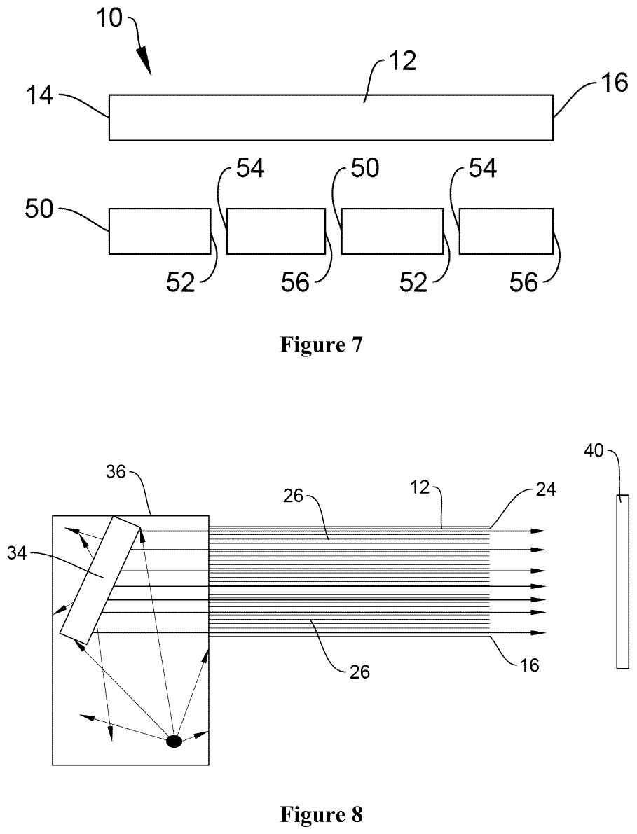

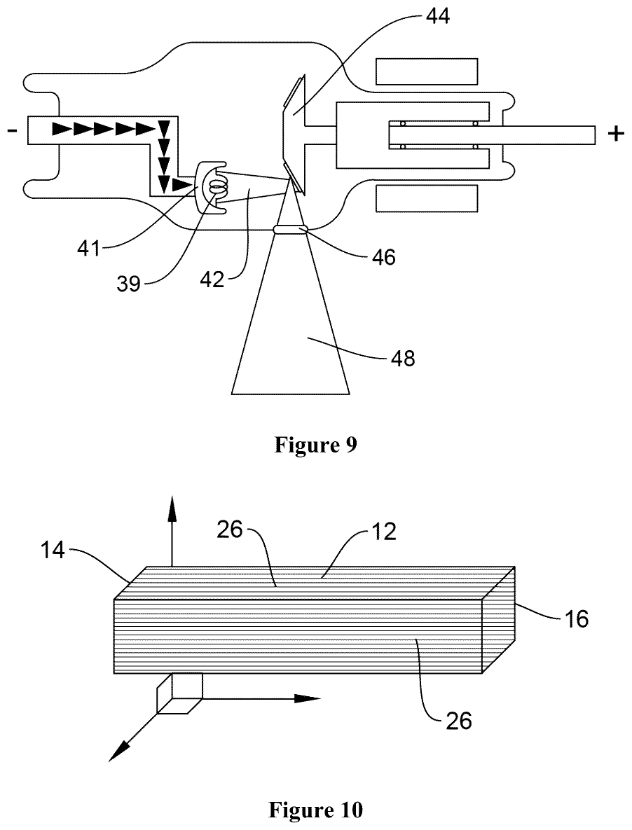

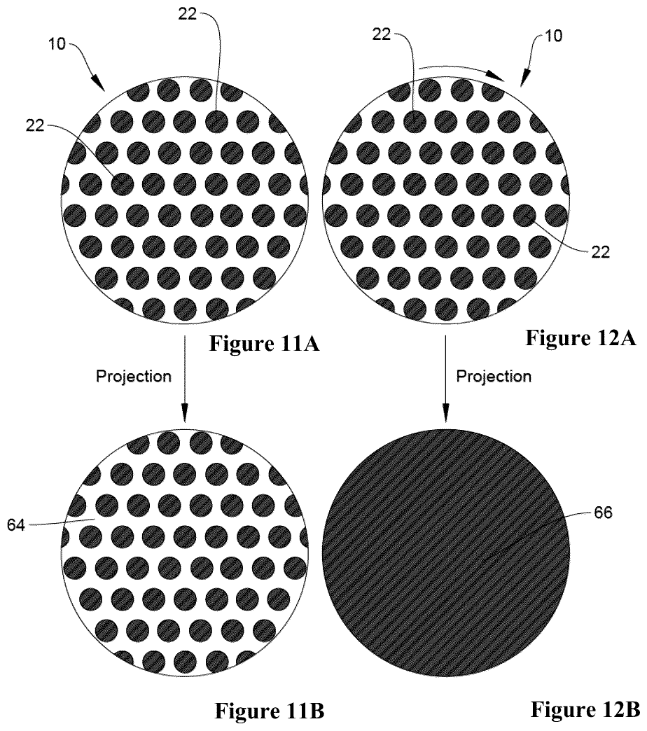

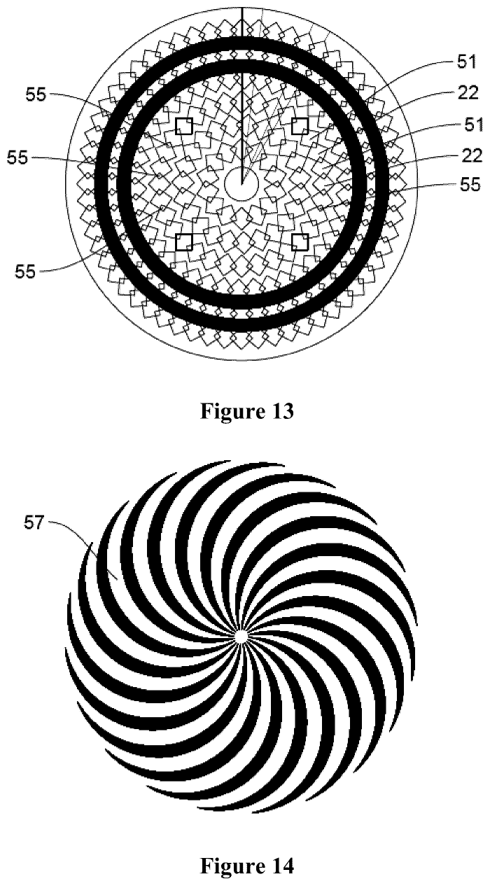

1-17. (canceled)

18. An electromagnetic radiation collimator configured for medical imaging comprising:
a radiopaque elongate housing having a first end and a second end, wherein:
the housing houses an array of parallel radiolucent beam paths, wherein each of the beam paths extend from an entrance aperture at the first end to a corresponding exit aperture at the second end, and
the housing is configured to engage a spinning motor for spinning or oscillating the housing;
wherein the first end is configured to receive electromagnetic radiation beams from an electromagnetic radiation source, and when the electromagnetic radiation beams enter the entrance aperture in line with the longitudinal axis of the respective beam path or at an angle less than or equal to a predetermined angle of deviation from the longitudinal axis of the respective beam path, the respective electromagnetic radiation beam traverses the length of the respective beam path such that the respective electromagnetic radiation beam is emitted from the corresponding exit aperture substantially parallel with the longitudinal axis of the beam paths and subsequently detected at an electromagnetic radiation detector.

19. The electromagnetic radiation collimator according to claim 18, wherein the array of parallel radiolucent beam paths is an array of parallel radiolucent tubes, wherein each of the tubes defines a lumen for providing the beam path, and wherein the radiolucent tubes are embedded in the elongate housing.

20. The electromagnetic radiation collimator according to claim 18, wherein the elongate housing is composed of a series of alternating first and second layers offset relative to each other, wherein the first layer comprises a first set of apertures each having a first shape, and the second layer comprises a second set of apertures each having a second shape, wherein the first shape and the second shape are partially intersecting such that the series of intersected shapes defines a lumen for providing the beam path.

21. The electromagnetic radiation collimator according to claim 18, wherein the elongate housing comprises at least a first elongated housing and a second elongated housing, wherein the first elongated housing has a length longer than the second elongated housing, the width of the entrance aperture of the first elongated housing is smaller than the width of the entrance aperture of the second elongated housing.

22. The electromagnetic radiation collimator according to claim 18, wherein the predetermined angle of deviation from the longitudinal axis of the beam path is less than 10°.

23. The electromagnetic radiation collimator according to claim 22, wherein the predetermined angle of deviation from the longitudinal axis of the beam path is less than 1°.

24. The electromagnetic radiation collimator according to claim 22, wherein the predetermined angle of deviation from the longitudinal axis of the beam path is 0°.

25. The electromagnetic radiation collimator according to claim 18, wherein the material of the elongate housing is one selected from the group of: aluminum, zirconia, titanium, and lead.

26. The electromagnetic radiation collimator according to claim 25, wherein the first end is perpendicular to the longitudinal axis of the beam paths.

27. The electromagnetic radiation collimator according to claim 18, wherein the elongate housing comprises at least a groove, wherein the grooves is adapted to engage to the spinning motor for spinning or oscillating the elongate housing.

28. The electromagnetic radiation collimator according to claim 27, wherein each of the grooves is 3 mm deep, and a width of 5 mm wide.

29. The electromagnetic radiation collimator according to claim 18, wherein the width of each beam path is 100 microns.

30. The electromagnetic radiation collimator according to claim 18, wherein the electromagnetic radiation source is one selected from the group of: X-ray radiation, and UV-C radiation.

31. The electromagnetic radiation collimator according to claim 30, wherein the width of the entrance and exit apertures for use with X-ray radiation are relatively smaller than width of the entrance and exit apertures for use with UV-C radiation.

32. An electromagnetic radiation imaging device adapted for use with two of the electromagnetic radiation collimators of claim 1, the device comprising:
an electromagnetic radiation source for generating electromagnetic radiation beams;
the first electromagnetic radiation collimator positioned between the electromagnetic radiation source and a subject region, such that the subject region is subjected to the substantially parallel electromagnetic radiation beams emitted from the exit apertures at the second end of the first electromagnetic radiation collimator;
the second electromagnetic radiation collimator positioned between the subject region and the electromagnetic radiation detector, wherein the second electromagnetic radiation collimator is adapted for filtering scattered radiation after propagating through the subject region such that propagated radiation, in line with the longitudinal axis of the beam path of the second collimator allows for the propagated radiation to be detected by the electromagnetic radiation detector.

33. The electromagnetic radiation image device according to claim 32, wherein the electromagnetic radiation source is a rotating anode X-ray source or a UV lamp.

34. The electromagnetic radiation image device according to claim 32, wherein the electromagnetic radiation detector is an X-ray film for X-ray radiation or a UV-C light sensor.

35. The electromagnetic radiation image device according to claim 33, wherein the electromagnetic radiation detector is an X-ray film for X-ray radiation or a UV-C light sensor.

36. The electromagnetic radiation image device according to claim 32, wherein a scatter medium is positioned between the electromagnetic radiation source and the first electromagnetic radiation collimator.
